(19) United States
(12) Patent Application Publication
(10) Pub. No.: US 2019/0024164 A1
(43) Pub. Date: Jan. 24, 2019

(54) ELECTRIC-FIELD-ASSISTED NUCLEOTIDE SEQUENCING METHODS

(71) Applicants: Paul SZAUTER, Albuquerque, NM (US); STC.UNM, Albuquerque, NM (US)

(72) Inventors: Payman Zarkesh-Ha, Albuquerque, NM (US); Jeremy Edwards, Albuquerque, NM (US); Paul Szauter, Albuquerque, NM (US)

(21) Appl. No.: 16/069,444

(22) PCT Filed: Jan. 11, 2017
(86) PCT No.: PCT/US2017/013042

§ 371 Date: Jul. 11, 2018

Related U.S. Application Data
(60) Provisional application No. 62/277,139, filed on Jan. 11, 2016.

(51) Int. Cl. C12Q 1/6874; G01N 27/414

(52) U.S. Cl. CPC C12Q 1/6874; G01N 27/4145

(57) ABSTRACT

This disclosure describes, in one aspect, a method of electric-field-assisted nucleotide sequencing. Generally, the method includes performing an ion-sensitive nucleotide sequencing method, applying an electric field across the device while the nucleotide sequencing reactions are being performed so that ions released by the sequencing reactions are directed to contact with the ion-sensitive detector, and detecting at least a portion of the released ions in contact with the ion-sensitive detector.

### CROSS-REFERENCE TO RELATED APPLICATION

This application claims priority to U.S. Provisional Patent Application No. 62/277,139, filed Jan. 11, 2016, which is incorporated herein by reference.

### SUMMARY

This disclosure describes, in one aspect, a method of electric-field-assisted nucleotide sequencing. Generally, the method includes performing an ion-sensitive nucleotide sequencing method, applying an electric field across the device while the nucleotide sequencing reactions are being performed so that ions released by the sequencing reactions are directed to contact with the ion-sensitive detector, and detecting at least a portion of the released ions in contact with the ion-sensitive detector.

In some embodiments, the method can further include reversing the electric field. directing detected ions away from the ion-sensitive detector, washing the released ions from the reaction site, and repeating the nucleotide sequencing steps to identify the next nucleotide base in the sequence.

In some embodiments, the ion-sensitive detector can include an ion-sensitive field effect transistor (ISFET) sensor or an avalanche ISFET sensor.

In some embodiments, the method can include detecting positively charged ions released by the nucleotide sequencing reactions.

In some embodiments, the method can include detecting negatively charged ions released by the nucleotide sequencing reactions.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Proton diffusion process during nucleotides incorporation on the DNA strands.

FIG. 2. Illustration of the effect of electric-filed assisted sequencing process.

FIG. 3. Illustration of the effect of electric-filed assisted flushing process.

### DETAILED DESCRIPTION OF ILLUSTRATIVE EMBODIMENTS

This disclosure describes an improved method for sequencing a polynucleotide. The method generally includes performing an ion-sensitive nucleotide sequencing method and applying an electric field across the device while the nucleotide sequencing reactions are being performed. The electric field directs ions that are released by the sequencing reactions to contact with an ion-sensitive detector. The sequence of the polynucleotide is deduced from the ion signals captured by the ion-sensitive detector.

Ion-sensitive field effect transistors (ISFETs) provide a non-optical-based nucleic acid sequencing technique that has good run times and high per-base accuracy. Due to their compatibility with standard complementary metal-oxide-semiconductor (CMOS) manufacturing process, ISFETs have become a common choice for many bio-sensing applications. For example, the core of an integrated circuit for non-optical genome sequencing is a large array of ISFET sensor elements fabricated using a low-cost CMOS integrated circuit process (US Patent Application Publication No. 2014/0234981 A1).

Arrays of ISFETs can be used in CMOS-compatible integrated circuits to directly perform DNA sequencing of genomes. For instance, arrays of 60-80 million ISFETs have been successfully fabricated into the ION TORRENT ION PI chip (Thermo Fisher Scientific, Waltham, Mass.). However, scaling up to arrays of 1 billion ISFETs per chip—the number needed for direct human genome sequencing—has become a great challenge due, at least in part, to the smaller well size and weaker signal generated by the ISFETs. Newer technologies, such as sequencing with oligonucleotides, also requires stronger detecting signals for more accurate sequencing.

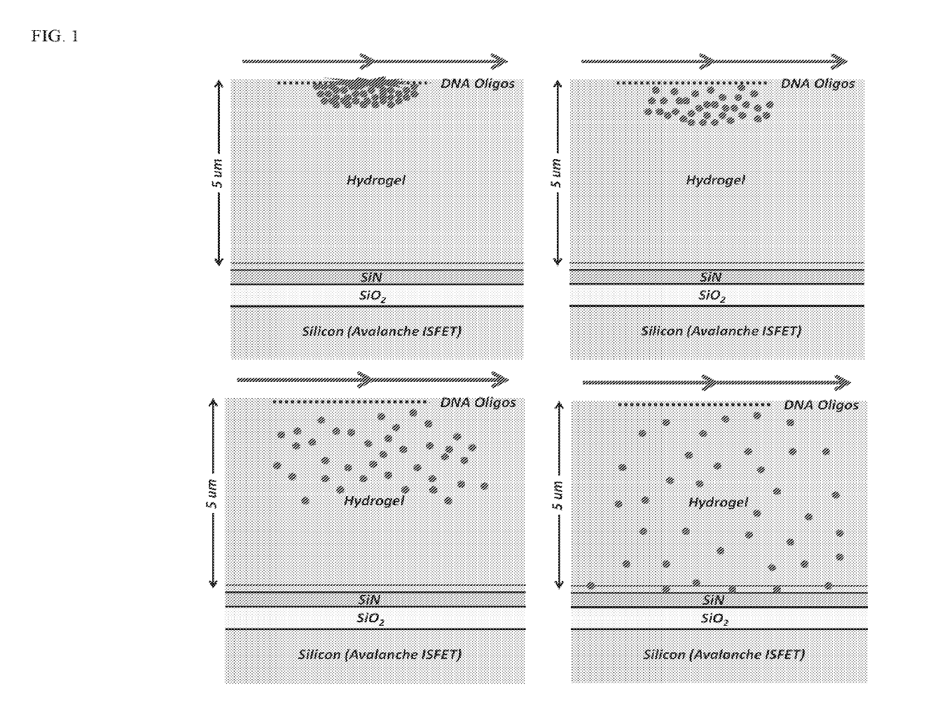

This disclosure describes electric-field-assisted DNA sequencing that can used in combination with any ion-sensitive nucleotide sequencing method such as, for example, ISFET-based nucleotide sequencing. FIG. 1 illustrates a cross section of an ion-sensitive DNA sequencing system. While the electric-field-assisted DNA sequencing is described herein in the context of an exemplary embodiment used with the sequencing method illustrated in FIG. 1, it may be used in the context of any ion-sensitive nucleotide sequencing method such as, for example, ISFET-based nucleotide sequencing technologies including, for example, those that use ION TORRENT chips.

The mechanism for detecting protons during the sequencing process involves protons diffusing and binding to the surface of an ion-sensitive detector, as shown in FIG. 1. As used herein, the word term “ion-sensitive sensor” or “ion-sensitive detector” refers to any device that is capable of detecting an ion such as, for example, a proton or a pyrophosphate ion. Exemplary ion-sensitive detectors include, for example, an ISFET sensor or an avalanche ISFET sensor. FIG. 1 illustrates that only a small percentage of available protons diffuse through the hydrogel and reach the surface of the ISFET sensor. The remainder of the protons end up distributed in the hydrogel or in the base fluid as illustrated in FIG. 1.

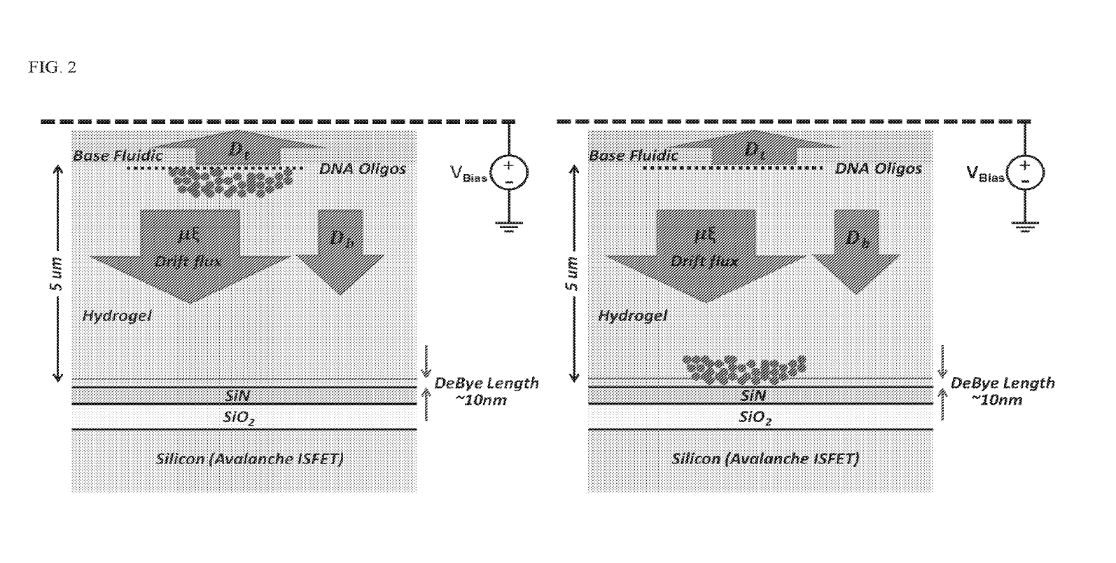

Electric-field-assisted nucleotide sequencing is illustrated in FIG. 2. An external electric filed is used to create proton flux toward the ion-sensitive detector such as, for example, the ISFET sensor as illustrated in FIG. 2. The electric-field-generated flux is stronger than the diffusion fluxes and, therefore, directs more of the protons to the ISFET sensor. The external electric field can be easily implemented by, for example, applying a positive potential on a grid or a solid metal (e.g., an electrode) on top of the device, with a negative potential on a grid or solid metal below the ion-sensitive detector. In some embodiments, the device substrate can serve as the electrode below the ion-sensitive detector, so that only one additional electrode is required. In such an embodiment, the electrode positioned above the device may be the only electrode that requires control in order to generate the electric field. Once the protons are on the ISFET sensor, the readout process can start.

The external electric filed directs the protons released during the sequencing reaction to the sensor, thereby reducing proton dilution in the fluid and/or hydrogel and, consequently, enhancing the ISFET signal. In addition, applying the electric field reduces the likelihood and/or extent to which protons can diffuse to a neighboring ISFET sensor, which reduces the crosstalk that typically occurs in conventional semiconductor-based sequencing techniques.

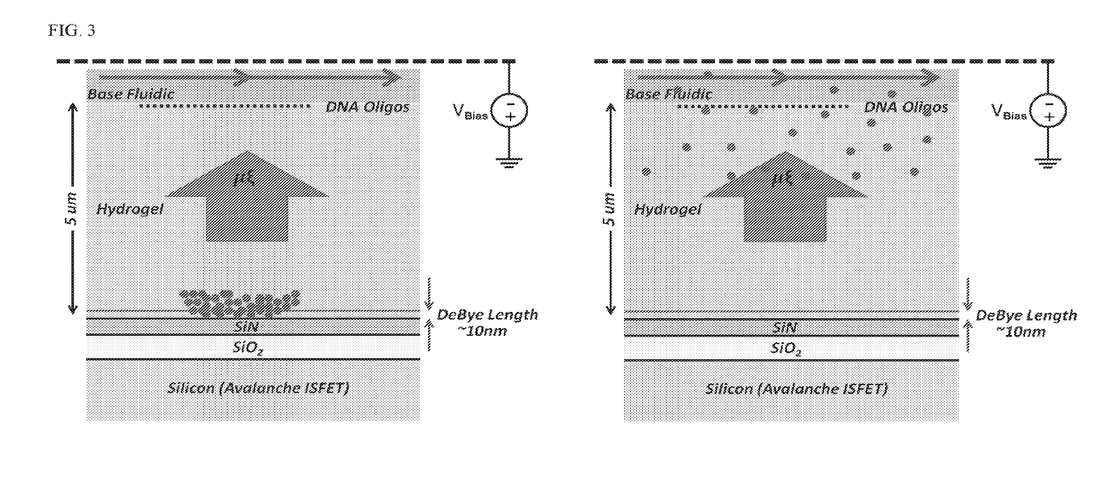

The external electric field also can also be used during flushing process in which the generated protons are removed from the ISFET sensor and flushed from the system before the next round of nucleotide incorporation. During the flush process, the electric field is reversed so that the negative potential is above the base fluid, directing protons away from the ISFET sensor and into the base fluid where the electrons can be flushed from the process, clearing the hydrogel for the next run. This process is shown in FIG. 3.

While described above in the context of an exemplary embodiment in which the ion released by the nucleotide sequencing synthesis reaction and detected by the system is a proton, the method described herein can be used in connection with a nucleotide sequencing method that involves detecting any ion released by the nucleotide sequencing reaction. The ion may be positively or negatively charged. The configuration illustrated in FIG. 2 or FIG. 3 may be used in connection with any nucleotide sequencing method that releases a positively charged ion such as, for example, a proton. Electric-filed-assisted sequencing also may be practiced using a configuration that is reversed with respect to the embodiment illustrated in FIG. 2 and FIG. 3 in order to detect a negatively charged ion (e.g., a pyrophosphate ion) released by the nucleotide sequencing method. That is, the electric field may be set up with a negative potential above the base fluid to drive the negatively charged ions toward the ion-sensitive detector. To flush the system, the electric field may be reversed so that a positive potential is generated above the base fluid drawing the negative ions off of the sensor and into the base fluid where the electrons can be flushed from the process, clearing the hydrogel for the next run.

Electric-field-assisted DNA sequencing can be used with any suitable ion semiconductor sequencing protocol. In one exemplary embodiment, the genomic DNA being sequenced may be made into a genomic library. The genomic library may be prepared by any one of several suitable conventional methods. For example, the genomic DNA can be fragmented by, for example, sonication to an average size of, for example, 100-160 bases, then ligated to unique forward and reverse adapters. The template pool can then be size selected to remove unincorporated primers. Size-selected libraries can be clonally amplified (e.g., on DNA-oligonucleotide beads) using, for example, emulsion PCR. Following amplification, template-carrying beads are separated from the reaction mixture by magnetic bead enrichment. Template-carrying beads are primed using oligonucleotides complementary to the adapters or hairpin ends. A DNA polymerase is added, and the beads are loaded onto the semiconductor sequencing chip where they bind to positively-charged wells.

As another example, described in U.S. Patent Application Publication No. US 20012/0270740 A1, DNA is fragmented by, for example, sonication to an average size of, for example, 200-400 bases, then ligated to hairpin ends. The template pool is then size selected to remove unincorporated primers. Size-selected libraries are clonally amplified by rolling circle replication to produce DNA nanoballs containing, for example, 3,000-5,000 copies of the original fragment. Template-carrying rolonies are primed using oligonucleotides complementary to the adapters or hairpin ends. A DNA polymerase is added, and the rolonies are loaded onto the semiconductor sequencing chip where they bind to positively-charged wells.

As yet another example, DNA is fragmented by, for example, sonication to an average size of, for example, 500 bases, then denatured and hybridized to an array of oligonucleotides designed against the target genome. In this method, no further priming is needed before a DNA polymerase is added.

Regardless of the method by which the genomic DNA library is prepared, sequencing is carried out by the addition of DNA nucleotides (e.g., at a concentration of 50 μm). One nucleotide (A, T, C, or G) is added during each cycle, followed by a wash with a suitable wash solution such as, for example, 6.4 mM MgCl₂, 13 mM NaCl, 0.1% Triton-X100 at pH 7.5.

Implementing the electric field assistance to the ion-sensitive nucleotide sequencing methods involves proper timing of applying the electric field. It is not possible to read an ion-sensitive sensor (e.g., ISFET, avalanche ISFET, or another type of ion detector) while an electric field is present. Thus, the electric field must therefore be applied prior to the reading of the sensors. Second, the electric field must be applied for a length of time sufficient to direct the ions to the sensors. If the electric field is not applied for a sufficient time, the electric field may not increase the sensitivity of ion detection sufficiently to be effective. Third, the electric field must have sufficient strength to direct the ions to the ion-sensitive detector. If the electric field is too weak, the electric field will be ineffective at directing the ions to the ion-sensitive detectors.

The strength of the external electric filed can depend, at least in part, on the physical structure and/or dimensions of the fluidic system. For example, if the thickness of the hydrogel is, for example, L=5 μm and the diffusivity of protons in the hydrogel is on the order of D≈10⁻⁹ m²/s, then it will take τ=L²/D (approximately 25 ms) for the protons to diffuse out of the hydrogel. The minimum electric field under such circumstances should be such that the protons move the 5 μm distance in less than the 25 ms in which they would diffuse from the hydrogel. Considering that the mobility of protons in hydrogel is about 3.6×10⁻⁷ m²/V·s and the electric field=velocity/mobility, then the minimum electric field will be approximately 500 V/m in the exemplary embodiment set forth above, but may vary somewhat depending, at least in part, on the parameters mentioned above.

Thus, the strength of the electric field can be a minimum of at least 200 V/m such as, for example, at least 250 V/m, at least 300 V/m, at least 350 V/m, at least 400 V/m, at least 450 V/m, at least 500 V/m, at least 550 V/m, at least 600 V/m, at least 650 V/m, at least 700 V/m, at least 750 V/m, at least 1000 V/m, at least 1250 V/m, at least 1500 V/m, at least 2000 V/m, at least 2500 V/m, at least 5000 V/m, at least 7500 V/m, at least 10,000 V/m, at least 12,500 V/m, at least 15,000 V/m, at least 17,500 V/m, at least 20,000 V/m, at least 22,500 V/m, or at least 25,000 V/m.

On the other hand, the current density due to the applied electric filed should be lower than 150 mA/cm² to decrease (or even eliminate) the likelihood and/or extent to which the hydrogel dissociates. Considering that the resistivity of hydrogel is about 20 Ωm and the electric field=current density x resistivity, then the maximum electric field will be approximately 30,000 V/m in the exemplary embodiment set forth above, but may vary somewhat depending, at least in part, on the composition of the hydrogel.

Accordingly, the strength of the electric field can be a maximum of no more than 50,000 V/m such as, for example, no more than 45,000 V/m, no more than 40,000 V/m, no more than 35,000 V/m, no more than 30,000 V/m, no more than 25,000 V/m, no more than 20,000 V/m, no more than 15,000 V/m, no more than 10,000 V/m, no more than 5000 V/m, no more than 2500 V/m, no more than 2000 V/m, no more than 1500 V/m, or no more than 1000 V/m.

In some embodiments, the strength of the electric field can fall within a range having endpoints defined by any minimum electric field strength listed above and any maximum electric field strength listed above that is greater than the minimum electric field strength. In some embodiments, for example, the strength of the electric field can be from 5000 V/m to 10,000 V/m.

Similarly, duration for applying the electric field depends, at least in part, on the system specifications. For instance, an ISFET sensor cannot be read while the external electric filed is applied. Therefore, the maximum duration will be limited to the nucleotide reaction rate, which can be in the order of several 100 ms. On the other hand, the duration of the electric field should be sufficient to transport free protons to the ISFET sensors, which depends, at least in part, on the strength of the applied electric field and/or the hydrogel thickness. Considering, that the mobility of protons in hydrogel is about 3.6×10⁻⁷ m²/V·s and an exemplary applied electric field of 30,000 V/m, then the minimum duration for the electric field will be approximately 0.5 ms when L=0.5 μm.

Thus, the applied electric filed can have a minimum duration of at least 0.1 ms such as, for example, at least 0.2 ms, at least 0.3 ms, at least 0.4 ms, at least 0.5 ms, at least 0.6 ms, at least 0.7 ms, at least 0.8 ms, at least 0.9 ms, at least 1.0 ms, at least 2.0 ms, at least 3.0 ms, at least 4.0 ms, at least 5.0 ms, at least 6.0 ms, at least 7.0 ms, at least 8.0 ms, at least 9.0 ms, at least 10 ms, at least 25 ms, at least 50 ms, at least 75 ms, at least 100 ms, at least 125 ms, at least 150 ms, at least 175 ms, at least 200 ms, at least 225 ms, at least 250 ms, at least 275 ms, at least 300 ms, at least 400 ms, or at least 500 ms.

The applied electric filed can have a maximum duration of no more than 1 second such as, for example, no more than 500 ms, no more than 250 ms, no more than 100 ms, no more than 90 ms, no more than 80 ms, no more than 70 ms, no more than 60 ms, no more than 50 ms, no more than 40 ms, no more than 30 ms, no more than 20 ms, or no more than 10 ms.

The applied electric field can have a duration within a range having as endpoints any minimum duration listed above and any maximum duration listed above that is greater than the minimum duration. For example, in some embodiments, the electric field may be applied for a duration of from 5 ms to 20 ms.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The particular examples, materials, amounts, and procedures described above are to be interpreted broadly in accordance with the scope and spirit of the invention as set forth in the claims below.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method comprising:
providing a device comprising an array of reaction sites, at least a portion of the reaction sites comprising an ion-sensitive detector;
adding a polynucleotide template and DNA sequencing reagents to at least a portion of the reaction sites comprising an ion-sensitive detector under condition effective to perform DNA sequencing reactions that release ions;
applying an electric field across the device while the DNA sequencing reactions are being performed such that the released ions are directed to contact with the ion-sensitive detector; and
detecting at least a portion of the released ions in contact with the ion-sensitive detector.

2. The method of claim 1 further comprising:
reversing the electric field directing detected ions away from the ion-sensitive detector;
washing the released ions from the reaction site;
adding fresh DNA sequencing reagents to the reaction site under condition effective to perform a DNA sequencing reaction that release ions;
applying an electric field across the device while the DNA sequencing reaction is being performed such that the released ions are directed to contact with the ion-sensitive detector; and
detecting at least a portion of the released ions in contact with the ion-sensitive detector.

3. The method of claim 1 wherein the ion-sensitive detector comprises an ion-sensitive field effect transistor (ISFET) sensor or an avalanche ISFET sensor.

4. The method of claim 1 wherein the released ions comprise positively charged ions.

5. The method of claim 4 wherein the positively charged ions comprise protons.

6. The method of claim 1 wherein the released ions comprise negatively charged ions.

7. The method of claim 6 wherein the negatively charged ions comprise pyrophosphate ions.
